(19) United States
(12) Patent Application Publication
(10) Pub. No.: US 2019/0000895 A1
(43) Pub. Date: Jan. 3, 2019

(54) COMPOSITION COMPRISING AT LEAST ONE PROBIOTIC BACTERIUM AND INTENDED TO BE USED FOR THE TREATMENT OF BUCCAL MYCOSES

(71) Applicant: VESALE PHARMA INTERNATIONAL, Pommerloch (LU)

(72) Inventors: Jehan Lienart Van Lidth De Jeude, Bigonville (LU); Johan Quintens, Hoegaarden (BE); Jean-Pol Warzée, Vieux-Genappe (BE)

(73) Assignee: VESALE PHARMA INTERNATIONAL, Pommerloch (LU)

(21) Appl. No.: 15/757,629

(22) PCT Filed: Sep. 8, 2016
(86) PCT No.: PCT/EP2016/071183

§ 371 Date: Mar. 5, 2018

(30) Foreign Application Priority Data
Sep. 8, 2015 (LU) 92820

(51) Int. Cl. A61K 35/747; A61K 9/00; A61P 31/10

(52) U.S. Cl. CPC A61K 35/747; A61K 9/0095; A61K 2035/115; A61P 31/10; A61K 9/0056

(57) ABSTRACT

The present invention relates to a composition which comprises at least one probiotic bacterium and which is intended to be used for the preventive and/or curative treatment of buccal mycoses, in particular intended to be used for the preventive and/or curative treatment of oral candidiasis, in particular oral candidiasis caused by the yeast Candida albicans, said at least one probiotic bacterium being the probiotic bacterium Lactobacillus rhamnosus No. LMG S-28148.

The present invention relates to a composition comprising at least one probiotic bacterium intended to be used for the preventive and/or curative treatment of buccal mycoses, in particular intended to be used for the preventive and/or curative treatment of oral candidiasis, in particular oral candidiasis caused by the yeast Candida albicans.

Buccal mycoses are frequent and are due to the excessive presence of yeast and fungi in the mouth. Among buccal mycoses, there are firstly acute or chronic oral candidiases and secondly deep mycoses of the mouth, such as for example cryptococcosis, mucomycosis, aspergillosis, histoplasmosis, paracoccidioidomycosis or coccidioidomycosis.

However, it is oral candidiases that are most frequent, being generally caused by a yeast of the Candida type (Candida tropicalis, Candida glabrata, Candida parapsilosis, Candida krusei, Candida stellatoidea, Candida pseudotropicalis, Candida famata, Candida rugosa, Candida geotrichium, Candida dubliniensis, Candida guiffiemondii and Candida albicans). Such yeasts are naturally present, normally, in the buccal flora without causing infection therein but may however, when they proliferate, cause mycosis.

The most frequent form of oral candidiasis is that caused more specifically by the yeast Candida albicans. This yeast is present naturally in approximately 35% to 50% of individuals but may cause mycosis of the mucous membrane of the mouth when it proliferates therein excessively to the point of becoming pathogenic and attacking the host tissues. This is a so-called opportunistic infection in that it is due to germs that are normally non-aggressive but which are liable to cause serious complications under the influence of local or systemic factors affecting for example the immune system. When the yeast Candida albicans gives rise to oral candidiasis, the formation of “thrush” in the mouth is commonly spoken of, which is responsible for an acidic, metallic, bitter or salty taste in the mouth and more rarely a sensation of burning in the mouth.

The prevalence of yeasts of the Candida type, that is to say the number of pathological cases caused by yeasts of the Candida type, varies according to numerous factors but a high prevalence has been observed during the summer months in women and in hospitalised individuals. A high proliferation of this yeast has also been noted in individuals consuming a diet rich in sugars, in individuals wearing a dental prosthesis, in individuals suffering from xerostomia (dry mouth), in persons consuming broad-spectrum antibiotics and/or corticoids and/or neuroleptics, in smokers, in immunodepressed individuals (for example because of HIV, diabetes, Down's syndrome or malnutrition) and in individuals suffering from cancer (cancer treatments such as chemotherapy and radiotherapy being factors favouring the development of these yeasts).

Age is also an important factor: a relatively low prevalence is observed in newborn babies and then increasing drastically in children before decreasing at an adult age. It should be noted that newborn babies despite everything frequently suffer from buccal mycoses and more particularly oral candidiasis since their immune system is not yet able to combat a proliferation of yeast of the Candida type, such as for example the yeast Candida albicans. Newborn babies (approximately 5% of them) acquire yeasts of the Candida type during their birth by transmission at the start of the vaginal canal of their mother. Aged persons also constitute persons at risk frequently subject to buccal mycoses therefore to oral candidiasis since they frequently combine several promoting factors, such as dry mouth and the taking of medicines.

Wearing a dental prosthesis and poor buccal hygiene, in particular wearing a dental prosthesis during the night, greatly promotes the proliferation of yeasts of the Candida type. This is because dental prostheses constitute a relatively acidic, moist and anaerobic environment (since the mucous membrane covered by the prosthesis is sheltered from oxygen and saliva), which is favourable to the development of yeasts of the Candida type

Moreover, poorly adjusted dental prostheses may damage the buccal mucous membrane, which increases the permeability thereof and facilitates an invasion of the tissues for example by the yeast Candida albicans. Damage to dental prostheses may also contribute to a proliferation of yeasts of the Candida type situated at the formation of the skin folds in the corners of the mouth, which forms intertriginous areas where another form of candidiasis, perleche, may develop.

Furthermore, species of Candida are capable of adhering to the surface of dental prostheses, the majority of which are manufactured from polymethacrylate, in which microcracks and cracks favourable to retention of these yeasts may form. It is also not rare for dental prostheses to be covered with a biofilm forming a veritable infection reservoir that may infect the mucous membrane. For this reason, disinfecting the prosthesis is an essential part of the treatment of oral candidiasis in persons who wear dental prostheses, and also good adjustment of the prosthesis in the mouth.

It is also recognised that the quantity and quality of the saliva constitute important oral defences against yeasts of the Candida type. This is because a reduction in the amount of saliva, a change in the composition of the saliva or hyposalivation are significant predisposing factors. Xerostomia (or dryness of the mouth) is often listed as being a major cause of oral candidiasis.

Malnutrition, related to poor absorption or bad food, but also deficiencies, in particular haematinic deficiencies (iron, vitamin B12, folic acid), may predispose to the appearance of oral candidiasis, by causing a reduction in the defences of the host and a reduction in the integrity of the epithelium. It would also appear that vitamin A or pyridoxine deficiencies are also responsible for oral candidiasis.

Some studies tend to demonstrate that nutrition rich in carbohydrates would also predispose to oral candidiasis. Studies carried out in vitro tend to demonstrate that yeasts of the Candida type develop and adhere (while being able to give rise to the formation of biofilm) more quickly in the presence of carbohydrates such as glucose, galactose and saccharose.

In smokers, tobacco addiction is an important predisposing factor even if, at the present time, the reasons for this relationship between oral candidiasis and tobacco addiction are unknown. One hypothesis is that cigarette smoke contains nutritional factors for Candida albicans or that local epithelial alterations occur and facilitate colonisation of species of Candida in the smoker.

It is also recognised that an imbalance in the buccal microbiota has a direct influence on the proliferation of yeast of the Candida type. As indicated above, broad-spectrum antibiotics, which eliminate competing bacteria and disturb the equilibrium between the naturally present oral microorganisms, in fact promote the development of oral candidiasis.

It should also be noted that the presence of some other lesions of the mucous membrane, in particular those that cause hyperkeratosis and/or dysplasia, predispose to a secondary infection such as oral candidiasis. Other physical impairments of the mucous membrane are sometimes associated with the proliferation of yeasts of the Candida type, such as for example fissures on the tongue or the presence of a piercing. It would also appear that women undergoing hormonal changes or taking contraceptive pills are more easily subject to oral candidiasis.

At the present time, buccal mycoses and more particularly oral candidiasis are mainly treated by means of local fungicides (such as nystatin, amphotericin B or miconazole) for a minimum treatment period of 10 to 15 days for acute forms and a minimum of 3 weeks for chronic forms.

However, another way of treating buccal mycoses, including oral candidiasis, is based on the use of probiotic bacteria, that is to say on the use of bacteria having a beneficial effect on health. It is in fact known that several probiotic bacterial strains are capable of breaking the biofilm formed by yeasts or that these probiotic bacteria have the ability to slow down the growth of these yeasts, in particular by modifying the pH of the surrounding environment or by affecting other mechanisms related to the development of yeasts, including in particular the development of yeasts of the Candida type.

In this regard, one publication discloses that probiotics of the Lactobacillus type, and more particularly the bacteria Lactobacillus rhamnosus GG (LGG), Lactobacillus casei Shirota, Lactobacillus reuteri SD2112, Lactobacillus brevis CD2, Lactobacillus bulgaricus LB86 and Lactobacillus bulgaricus LB Lact act as inhibiters of the growth of yeasts of the Candida type and therefore make it possible potentially to treat this pathogenic yeast when it proliferates abnormally, that is to say to treat oral candidiasis (Jiang et al., 2015. Benef. Microbes, 6(3): 361-368).

Another document indicates the inhibiting effect of a combination of the probiotics Lactobacillus casei and Bifidobacterium breve on the development of yeasts of the Candida type, this mixture of probiotics being administered in the form of a drink (yogurt) to elderly patients suffering from oral candidiasis. According to the results of this study, approximately 57% of the patients treated were able to have the populations of Candida significantly reduced while total elimination of this yeast was observed only in approximately 12% of the patients (Mendonça et al., 2012. J. Dent. Braz., 23(5): 534-538).

Another clinical study carried out in vivo describes that a reduction in the number of cases (reduction in the prevalence) of oral candidiasis due to yeasts of the Candida type is observed in elderly persons consuming the probiotic Lactobacillus rhamnosus GG (LGG) daily. This study indicates that, after 16 weeks, the prevalence of yeasts of the Candida type is reduced by some 32% as against an increase of 28% in the placebo group (Hatakka et al., 2007. J. Dent. Res., 86(2): 125-130).

The document WO 99/17788 discloses a method for treating infections caused by Candida, in particular in the mouth. More particularly, this document describes tests carried out on mice to which various probiotics were administered by enteral route in the form of a lyophilised powder. The probiotics tested are L. reuteri, B. infantis, B. lactis, L. acidophilus and L. casei and this document envisages that these may be associated with one or more other probiotics, including L. casei rhamnosus and L. rhamnosus. It should be noted that this document demonstrates that the strain L. casei GG (LGG) is not effective in reducing the incidence of Candida.

The document EP 2455095 relates to a therapeutic use of a probiotic of the type L. rhamnosus for treating dental oral infections. The tests carried out in this prior document by oral administration of the strain L. rhamnosus KO3 as a probiotic show that the latter does not make it possible to ensure total elimination of Candida.

The document of Ishikawa et al. (Journal of Prosthodontics, 24, 194-199, 2015) relates to an in vivo study carried out for evaluating treatment of oral candidiasis by means of a combination of probiotics, namely L. acidophilus HS101, B. bifidum and L. rhamnosus HS111 administered together in lyophilised form in a capsule, at the rate of one capsule per day for 5 weeks. Unfortunately, as is clear from this scientific article, this combination of probiotics does not make it possible to ensure total elimination of Candida even after 5 weeks of treatment.

The document of Hasslöf et al. (BMC Oral Health, 10(18), 2010) relates to an in vitro study testing the efficacy of various probiotics, including L. rhamnosus LB21 and L. rhamnosus LGG for treating/inhibiting the growth of Candida albicans. It should be noted that this prior document envisages that these probiotics be administered in vivo while being formulated in yogurt but that such a formulation is certainly not suitable since the document of Mendonça et al. shows that a formulation of Lactobacillus in a yogurt is far from being effective.

The document of Mändar (Oral Microbiology Immunology, 23, 130-147, 2008) also relates to an in vitro study testing various probiotics on various pathogenic bacteria, including the probiotic L. rhamnosus, on the pathogen Candida albicans and shows that this probiotic has no effect on Candida albicans issuing from saliva.

Unfortunately, at the present time, these treatments by means of probiotic bacteria prove to be of low performance, which restricts the clinical use of these probiotics on a large scale for treating buccal mycoses including acute or chronic oral candidiasis. This lack of efficacy of the treatments also restricts the use of these probiotics for prophylactic (preventive) treatment of buccal mycoses including oral candidiases. This is because, as is clear from the studies mentioned above, the majority of the current compositions based on probiotic bacteria only slightly reduce the presence of pathogens (yeasts, fungi, etc.) in vivo, such as for example the presence of yeasts of the Candida type proliferating excessively in the mouth. These studies also indicate that the current compositions do not completely eliminate these yeasts except in a very small proportion of the individuals treated. Moreover, the durations of treatment are relatively long with the current compositions comprising probiotic bacteria.

There therefore exists, at the present time, in order to propose a treatment alternative to the use of fungicides, a real need to procure a composition based on at least one probiotic bacterium that makes it possible to treat buccal mycoses effectively and quickly, preventatively and/or curatively, in particular acute or chronic oral candidiasis, in particular oral candidiases related to the proliferation of the yeast Candida albicans.

The aim of the invention is to overcome the drawbacks of the prior art by procuring a composition as indicated at the start, characterised in that said at least one probiotic bacterium is the probiotic bacterium Lactobacillus rhamnosus No. LMG S-28148.

In the context of the present invention, it has been shown, surprisingly, that this specific strain No. LMG S-28148 of Lactobacillus rhamnosus, filed with the BCCM (Belgian Coordinated Collections of Microorganisms) and the LMG (Laboratorium voor Microbiologie—Bacteriënverzameling) on 27 Jan. 2014 under the Budapest Treaty makes it possible to obtain results superior to those recorded during a treatment with the bacterial strains currently used, during a preventive or curative treatment of buccal mycoses, in particular for preventive and/or curative treatment of an acute or chronic oral candidiasis.

The term “acute oral candidiasis” means that this most frequent form of candidiasis is due to local factors.

The term “chronic oral candidiasis” means that this form of candidiasis, which is more rare, is mainly but not exclusively due to a deficit in the immune system.

Moreover, it has been determined, in the context of the present invention, that a composition comprising the probiotic Lactobacillus rhamnosus No. LMG S-28148 makes it possible to treat acute or chronic buccal mycoses, in particular acute oral candidiases, in only a few days. This is because it has been observed, surprisingly, that a composition according to the invention is effective and provides treatment and elimination of buccal mycoses, in particular treatment and elimination of acute oral candidiases, in an average period of only 3 to 5 days or even 7 days, whereas current treatments based on formulations including probiotics require a treatment period of at least 15 days or even several weeks without however guaranteeing effective treatment. This is particularly advantageous since, even in the case of acute oral candidiases, a composition according to the invention makes it possible to quickly, that is to say in 2 or 3 days or even 5 days and at a maximum in one week, stop any proliferation of yeasts of the Candida type, in particular any proliferation of the yeast Candida albicans, and to completely treat a buccal mycosis such as for example an acute oral candidiasis.

Advantageously, the composition according to the invention further comprises at least one additional probiotic chosen from the group consisting of the following probiotics: Archaea, Firmicutes, Bacteroidetes, Proteobacteria, Actinobacteria, Verrucomicrobia, Fusobacteria, Metanobacteria, Spirochaetes, Fibrobacters, Deferribacteres, Deinococcus, Thermus, Lactobacillus, Cyanobacteria, Methanobrevibacterium, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae, Faecalibacterium, Bacteroides, Parabacteroides, Prevotella, Eubacterium, Akkermansia, Bacillus, Butyrivibrio and Clostridium, or a combination thereof.

Preferably the composition according to the invention is in an orosoluble or orodispersible form. Such a form, which dissolves orally, that is to say when it is sucked or by simple contact with saliva, is preferred according to the present invention since it has been shown that this form of a composition according to the invention makes it possible not only to guarantee a sufficient contact time of the probiotic bacteria with the pathogenic yeasts or fungi, such as for example yeasts of the Candida type (for example the yeast Candida albicans) but also that this contact does indeed take place throughout the mouth, that is to say over the whole of the surface of the mouth tissues. For example, the composition according to the invention may be in the form of a dry orosoluble powder that will mix with the saliva and therefore be dispersed quickly and homogeneously in the mouth.

Preferentially, said orosoluble or orodispersible form of a composition according to the invention solubilises or disperses in a few seconds, for example in 15 seconds, preferably in 10 seconds, preferentially in 5 seconds, in a preferred fashion in 2 to 3 seconds. These periods have been determined as being suitable so that the probiotic bacterium Lactobacillus rhamnosus No. LMG S-28148 and optionally one or more other additional probiotic bacteria of the composition according to the invention can provide optimum treatment of buccal mycoses, for example of oral candidiasis, by acting on the yeast Candida albicans, the proliferation of which is blocked and treatment (elimination) assured.

Advantageously, said orosoluble or orodispersible form of a composition according to the invention is a powder in a sachet or not, a cachet or a tablet. Any other suitable orosoluble or orodispersible form is also included in the scope of the present invention.

Preferably, the composition according to the invention is in the form of a neat syrup. The term “neat syrup” designates, within the meaning of the present invention, a form of the composition according to the invention which, once put in solution in a liquid or semi-liquid vehicle, makes it possible to form a liquid dose for oral administration.

Preferentially, the composition according to the invention is in a liquid or semi-liquid form.

Advantageously, said liquid or semi-liquid form of a composition according to the invention is a suspension, a dispersion, a gel, a paste, a syrup, a toothpaste, a chewing gum, a potable liquid, a mouthwash, an emulsion or a microemulsion.

Preferably, said liquid or semi-liquid form of a composition according to the invention is an aqueous or oily form.

Advantageously, the composition according to the invention further comprises additional excipients chosen from the group consisting of gums, cellulose, acrylic derivatives, thixotropic agents, stabilisers, preservatives and mixtures thereof.

Preferentially, the composition according to the invention further comprises one or more polymers and/or one or more compounds for conferring viscoelastic properties.

Such additional excipients and such polymers or compounds conferring viscoelastic properties may make it possible to confer suitable properties on the composition according to the invention, for example a suitable viscosity enabling the composition to be distributed throughout the mouth, that is to say to come into contact with all the mouth tissues, so that the probiotic bacterium Lactobacillus rhamnosus No. LMG S-28148 and optionally one or more other additional probiotic bacteria of the composition according to the invention can provide optimum treatment of buccal mycoses, for example oral candidiasis, by acting then on the yeast Candida albicans, the proliferation of which is blocked and treatment (elimination) assured.

Other embodiments of a composition according to the invention are indicated in the accompanying claims.

### EXAMPLES

Prospective In Vivo Clinical Study: Evaluation of the Efficacy and Tolerance of Lactobacillus Rhamnosus No. LMG S-28148 in Elderly Patients Suffering from “Thrush” Caused by Candida Albicans

Eleven patients (8 women and 3 men) in whom buccolingual mycosis was detected (clinical diagnosis) were subjected for 7 days to a daily administration of Lactobacillus rhamnosus No. LMG S-28148 at the rate of 3 times per day (after each meal—morning, midday and evening). More specifically, for this study, the administration method consisted of 3 doses of orosoluble sachets each containing 5.10⁹ CFU/sachet of the probiotic Lactobacillus rhamnosus No. LMG S-28148.

In order to judge the efficacy and tolerance of Lactobacillus rhamnosus No. LMG S-28148 in these patients, photographs were compared before and after treatment. In addition, the changes in the symptoms (pain, dryness, dysgeusia) and any secondary effects were noted.

After 7 days of treatment, a cure was considered to be “complete” if all the clinical symptoms were eliminated: elimination of pain, buccal dryness, dysgeusia and visible disappearance of the infection (that is to say disappearance of whitish filaments and deposits), and also the disappearance of a metallic taste in the mouth.

Table 1 below compiles the results obtained through this prospective in vivo clinical study.

TABLE 1

| | Sex | | |
|---|---|---|---|
| Patient | M/F | Age | Cure after 7 days |
| 1 | F | 82 | Complete and absence of secondary effects |
| 2 | F | 91 | Complete and absence of secondary effects |
| 3 | F | 82 | Complete and absence of secondary effects |
| 4 | F | 72 | Complete and absence of secondary effects |
| 5 | M | 92 | Complete and absence of secondary effects |
| 6 | F | 87 | Complete and absence of secondary effects |
| 7 | M | 80 | Complete and absence of secondary effects |
| 8 | F | 76 | No cure but irregular dosage because of |
| | | | difficult compliance of the patient |
| 9 | F | 87 | Complete and absence of secondary effects |
| 10 | M | 90 | Complete and absence of secondary effects |
| 11 | F | 87 | Complete and absence of secondary effects |

As can be seen, with the exception of the patient who did not comply with the daily dose at the rate of 3 orosoluble sachets each containing 5.10⁹ CFU/sachet of the probiotic Lactobacillus rhamnosus No. LMG S-28148, all the patients were treated effectively since, in all cases, a “complete” cure was observed after only 7 days of treatment. It should also be noted that, for all the patients, no secondary effect was noted.

Naturally the present invention is in no way limited to the embodiments described above and many modifications can be made thereto without departing from the scope of the accompanying claims.

1. A composition comprising at least one probiotic bacterium and being intended to be used for the preventive and/or curative treatment of buccal mycoses, in particular intended to be used for the preventive and/or curative treatment of oral candidiasis, in particular oral candidiasis caused by the yeast Candida albicans, said composition being characterised in that said at least one probiotic bacterium is the probiotic bacterium Lactobacillus rhamnosus No. LMG S-28148.

2. The composition according to claim 1, further comprising at least one additional probiotic chosen from the group consisting of the following probiotics: Archaea, Firmicutes, Bacteroidetes, Proteobacteria, Actinobacteria, Verrucomicrobia, Fusobacteria, Metanobacteria, Spirochaetes, Fibrobacters, Deferribacteres, Deinococcus, Thermus, Lactobacillus, Cyanobacteria, Methanobrevibacterium, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae, Faecalibacterium, Bacteroides, Parabacteroides, Prevotella, Eubacterium, Akkermansia, Bacillus, Butyrivibrio and Clostridium, or a combination thereof.

3. The composition according to claim 1, wherein said composition is in an orosoluble or orodispersible form.

4. The composition according to claim 3, wherein said orosoluble or orodispersible form solubilises or disperses in a period of a few seconds.

5. The composition according to claim 3, wherein said orosoluble or orodispersible form is a powder in a sachet or not, a cachet or a tablet.

6. The composition according to claim 1, wherein said composition is in the form of a neat syrup.

7. The composition according to claim 1, wherein said composition is in a liquid or semi-liquid form.

8. The composition according to claim 7, wherein said liquid or semi-liquid form is a suspension, a dispersion, a gel, a paste, a syrup, a toothpaste, a chewing gum, a potable liquid, a mouthwash, an emulsion or a microemulsion.

9. The composition according to claim 7, wherein said liquid or semi-liquid form is an aqueous or oily form.

10. The composition according to claim 1, further comprising one or more additional excipients selected from the group consisting of gums, cellulose, acrylic derivatives, thixotropic agents, stabilisers, preservatives and mixtures thereof.

11. The composition according to claim 1, further comprising one or more polymers and/or one or more compounds for conferring viscoelastic properties.
